(19) United States
(12) Patent Application Publication
(10) Pub. No.: US 2025/0326187 A1
(43) Pub. Date: Oct. 23, 2025

(54) FEEDSTOCKS COMPRISING HYPHAE, PRODUCTS PRODUCED THEREFROM, AND METHODS OF FORMING PRODUCTS THEREFROM

(71) Applicant: MUSHROOM MATERIAL PRIVATE LIMITED, Singapore (SG)

(72) Inventors: Paul Malcolm Guinibert, Auckland (NZ); Shaun Edward William Seaman, Auckland (NZ); Jotinder Pal Singh, Auckland (NZ)

(21) Appl. No.: 18/870,057

(22) PCT Filed: May 31, 2023
(86) PCT No.: PCT/NZ2023/050057

§ 371 Date: Nov. 27, 2024

(30) Foreign Application Priority Data
Jun. 1, 2022 (NZ) 788942

(51) Int. Cl. B29C 70/02

(52) U.S. Cl. CPC B29C 70/021; B29K 2911/10

(57) ABSTRACT

A feedstock for moulding may include a plurality of feed components. The plurality of feed components may include hyphae. The feedstock may further include a binder. The plurality of feed components may be coated by and/or impregnated with the binder. The binder may be suitable to bind the plurality of feed components together when activated. Methods include preparing the feedstocks and for preparing products formed therefrom.

### FIELD

This invention relates to feedstocks for moulding comprising hyphae and products produced therefrom, methods of moulding said feedstocks, and methods of preparing said feedstocks.

### BACKGROUND

Mycelium-based materials can be used to mould products. The raw materials can comprise spawn which may require a pre-growth phase over multiple days. After the pre-growth is complete, the spawn is then placed into a mould where mycelium grows over another multi-day period into a formed shape defined by the mould. The amount of time between the preparation of the spawn and production of the formed product can therefore be considerable. Requiring the spawn to grow within the mould over multiple days also severely limits throughput.

Mycelium-colonised particles and/or fibres can be used as a feedstock with heated press moulds. When the feedstock is pressed within the mould, water within the feedstock evaporates and saturates glucans which naturally occur within fungal cell walls. This causes the glucans to flow like a resin to bind the feedstock into a formed shape.

Although a particle/fibre-based feedstock may offer improved throughput compared to feedstocks which require a period of growth within the mould, particle/fibre-based feedstocks also suffer from several drawbacks. For example, they must necessarily contain a moisture content of at least 40% so that, when moulded in a heated platen press, a sufficient amount of steam is generated to cause the glucans to flow. This moisture content significantly increases the overall density of the feedstock and makes shipping and handling of the feedstock more expensive due to an increased weight. The high moisture content can also allow for bacteria or other fungi to colonise and contaminate the feedstock, thereby limiting the shelf life of the particle/fibre-based feedstock.

While the particle/fibre-based feedstock can be dehydrated and used with a steam-injecting heated platen press, this can also pose drawbacks. Firstly, requiring the injection of steam into the heated platen press restricts the number and variety of moulds that the particle/fibre-based feedstock is compatible with. Secondly, although the feedstock may be initially dehydrated, it will immediately begin to absorb moisture from the atmosphere after the dehydration process. The feedstock will therefore gradually become more dense and more hospitable to contaminating bacteria or fungi, thereby increasing shipping/handling expenses and reducing the shelf life of the feedstock.

### SUMMARY

In some configurations, a feedstock for moulding can comprise a plurality of feed components comprising hyphae; and a binder; wherein: the plurality of feed components is coated by and/or impregnated with the binder, and the binder is suitable to bind the plurality of feed components together when activated.

Further configurations can be implemented according to any one of the dependent claims.

In further configurations, a method of forming a product can comprise providing a feedstock to a mould, the feedstock comprising a plurality of feed components comprising hyphae, the plurality of feed components being coated by and/or impregnated with a binder, moulding the feedstock, and before, after, or during the moulding of the feedstock, activating the binder to bind the plurality of feed components together.

Further configurations can be implemented according to any one of the dependent claims.

In further configurations, a method of producing a feedstock for moulding can comprise preparing a plurality of feed components comprising hyphae, and coating and/or impregnating the plurality of feed components with a binder; wherein the binder is suitable to bind the plurality of feed components together when activated.

Further configurations can be implemented according to any one of the dependent claims.

In further configurations, a method of producing a feedstock for moulding can comprise colonising a growth substrate with hyphae, the growth substrate comprising a binder distributed throughout the growth substrate; and processing the colonised growth substrate to produce a plurality of feed components; wherein the binder is distributed throughout each feed component, and wherein the binder is suitable to bind the plurality of feed components together when activated.

Further configurations can be implemented according to any one of the dependent claims.

In further configurations, a method of producing a feedstock for moulding can comprise preparing a plurality of feed components by colonising a plurality of growth substrates with hyphae, each growth substrate comprising a binder distributed throughout the growth substrate; wherein the binder is distributed throughout each feed component, and wherein the binder is suitable to bind the plurality of feed components together when activated.

Further configurations can be implemented according to any one of the dependent claims.

In further configurations, a method for continuous production of a feed component can comprise providing a growth material to an extrusion line, colonising the growth material with a fungal inoculum, with the colonised growth material remaining in-line, growing hyphae throughout the colonised growth material to produce a feed component material, and extruding the feed component material.

Further configurations can be implemented according to any one of the dependent claims.

In further configurations, a formed product at least partially moulded from a feedstock can comprise: a plurality of feed components comprising hyphae, and a binder; wherein the plurality of feed components is bound together by the binder.

Further configurations can be implemented according to any one of the dependent claims.

It is acknowledged that the terms “comprise”, “comprises” and “comprising” may, under varying jurisdictions, be attributed with either an exclusive or an inclusive meaning. For the purpose of this specification, and unless otherwise noted, these terms are intended to have an inclusive meaning—i.e., they will be taken to mean an inclusion of the listed components which the use directly references, and possibly also of other non-specified components or elements.

Reference to any document in this specification does not constitute an admission that it is prior art, validly combinable with other documents or that it forms part of the common general knowledge.

### BRIEF DESCRIPTION OF THE DRAWINGS

The accompanying drawings which are incorporated in and constitute part of the specification, illustrate examples of the invention and, together with the general description of the invention given above, and the detailed description of examples given below, serve to explain the principles of the invention, in which:

FIG. 1 depicts a schematic illustration of a feedstock for moulding.

FIG. 2 depicts a schematic illustration of the feedstock of FIG. 1 during moulding.

FIG. 3 depicts an example of a method of moulding a feedstock.

FIG. 4 depicts an example of a method of preparing a feedstock.

FIG. 5 depicts a further example of a method of preparing a feedstock.

FIG. 6 depicts a further example of a method of preparing a feedstock.

FIG. 7 depicts an example of an extrusion system for the continuous production of a feedstock.

### DETAILED DESCRIPTION

### Feedstocks for Moulding

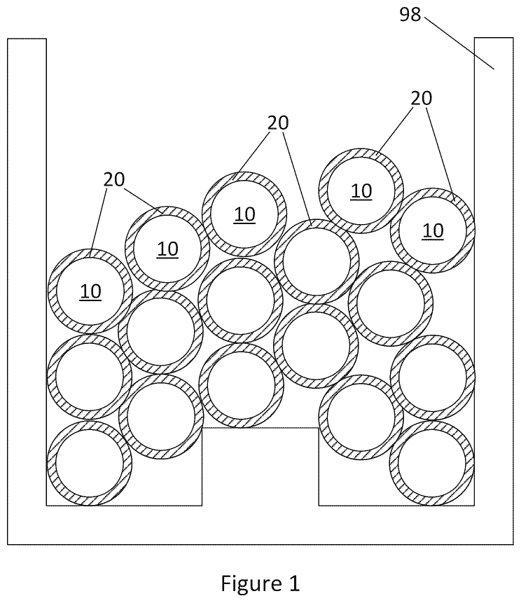

FIG. 1 depicts a schematic illustration of an example of a feedstock 1 for moulding. The feedstock 1 can comprise a plurality of feed components 10 which comprise hyphae. The feedstock 1 can further comprise a binder 20. The plurality of feed components 10 can be coated by and/or impregnated with the binder 20, which can be suitable to bind the plurality of feed components 10 when activated. The feedstock 1 is depicted has having been placed into a mould 98 for forming.

In this particular illustration, binder 20 is schematically represented as an exaggerated coating about each feed component 10, although it should be understood that the respective scales of the feed component 10 and coating of binder 20 may differ. Furthermore, in other examples, the feed components 10 may have little to no coating of binder 20, with the binder 20 instead being substantially or completely impregnated within each feed component 10.

The feedstock 1 can be suited to be moulded into a formed shape, with the individual plurality of feed components bound together by the binder once the binder is activated. The binder can be activated before, during, or after the moulding of the feedstock 1, as described in more detail herein.

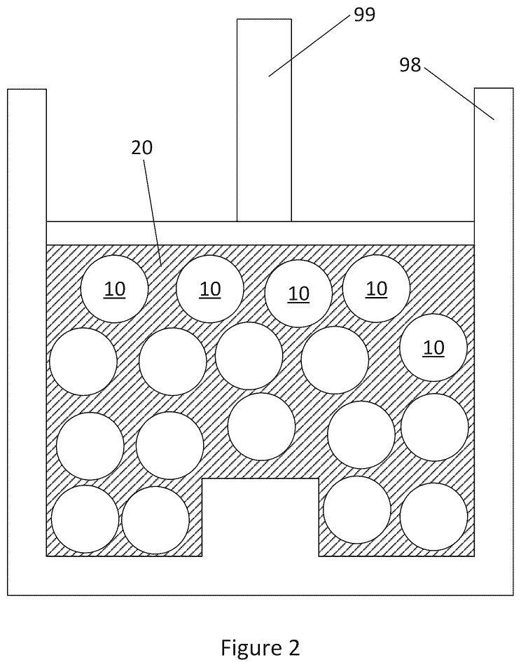

FIG. 2 depicts a schematic illustration of the feedstock after the binder 20 has been activated in mould 98. Platen 99 has formed the feedstock into the shape defined by the mould. In this particular illustration, the activated binder 20 is depicted as a matrix within which the feed components 10 are embedded. It should be understood that this is exaggerated for illustrative purposes and is not limiting; while the activated binder 20 can form a matrix in some examples of the feedstock, the relative dimensions and/or volumes of the activated binder 20 and feed components 10 can differ. In other examples, the activated binder 20 may not substantially form a matrix after activation (for example, if the binder 20 does not melt or flow upon activation.)

The feed components 10 are typically discrete components comprising a growth substrate which has been intentionally colonised with one or more fungi. The colonising fungus or fungi can grow such that the feed component comprises mycelium or hyphae. Several example methods for producing feed components are described in more detail herein.

In the schematic illustrations of FIGS. 1 and 2, feed components 10 are generally pellet-shaped and may have diameters on the order of 0.1 cm to 1 cm. However, it should be understood that this is not a limitation. The shape and or/size of the feed components 10 can vary depending on the shape and/or size of the final formed product, the size and/or shape of the mould, the nature of the substrate used to produce the feed components 10, and other factors. For example, if the substrate comprises discrete components (e.g. wood chips), then the size of those discrete components can determine the minimum size of the feed components 10.

In some examples, feed components 10 can take the form of pellets, granules, powder, sheets, blocks, chips, balls, particles, fragments, shavings, and/or flakes. The shape and size of the feed components 10 can depend at least on the desired application of feedstock 1.

For example, if the formed product which will be produced through the moulding process has complex features which require high resolution, then the feed components 10 can be small pellets or granules such that when the feedstock 1 is moulded, the dimensions of the feed components 10 are such that the features are adequately resolved.

In another examples, if the formed product is a flat planar structure (e.g. sheet material for building or insulation applications), then the feed components 10 may be sheets of material. In still further examples, the feed components 10 of feedstock 1 can have any combination of different shapes and sizes. For example, the feed components 10 can include planar sheets of material in addition to granules or pellets.

### Binders

The binder 20 can bind the feed components 10 together once activated. For example, the binder 20 can be crosslinked and/or thermoset upon activation. In other examples, the binder 20 can be or act as a hot melt adhesive. The binder 20 can also affect the material properties of the feed component 10 and/or of the formed product once the feedstock 1 has been moulded. Example binders 20 can include thermoplastics (such as PBAT or PETG), PLA, PBS, PPS, biopolyethylene, and bioplastics.

The feed components 10 can be coated with and/or impregnated by the binder 20. In some examples, the feed components 10 can be prepared without any initial binder present in the growth substrate and can be subsequently post-processed to coat and/or impregnate the feed components 10 with binder 20. For example, the binder 20 can be applied to the feed components 10 by spraying, rolling, submerging, or dousing the feed components 10 with the binder 20.

The duration of the treatment of the feed components 10, the amount of binder 20 used in the treatment process, and the nature of the feed components 10 and binder 20 (e.g. their absorbency and viscosity, respectively) can at least partially determine the extent to which the feed components 10 are impregnated with binder 20 (and/or the extent to which the feed components 10 are coated by binder 20.) In some examples, the feed components 10 can have substantially no coating, with the majority or entirety of the binder 20 impregnated within the feed components 10.

For example, the feed components 10 can be treated by lightly spraying the feed components with binder 20, such that the treated feed components 10 are only lightly or partially coated by binder 20. In other examples, the feed components 10 can be doused in a sufficient quantity of binder 20 for a sufficient duration such that the binder 20 soaks through a substantial portion of the feed components 10 and/or is applied as a relatively thick coating on feed components 10. The feed components 10 can also or alternatively be coated in a sufficiently thick layer to effectively create a matrix of binder 20 into which the feed components 10 are embedded.

In other examples, the feed components 10 may not be separately treated with binder 20, and can be prepared with binder 20 already dispersed throughout the feed components 10. For example, the binder 20 can initially be incorporated into the growth substrate which can be colonised by fungus to create the feed components 10, as described herein.

Furthermore, in some examples, a combination of multiple types of binder 20 can be used. A combination of treatment processes (e.g. spraying, rolling, dousing) can also be used to coat and/or impregnate the feed components 10 with the binder 20 (or multiple binders).

The specifics of the activation of the binder 20 can depend on the selection and properties of the binder 20. These can depend on the requirements of the feedstock 1 and/or the corresponding products formed from the feedstock 1.

In some examples, the binder 20 can be chosen so that it is activated by the application of heat. For example, the binder 20 can be a thermoplastic that melts and activates at a sufficient temperature, and the corresponding feedstock 1 can be used in conjunction with a heated mould. The binder 20 can then be activated during the moulding of the feedstock 1 in the heated mould, thereby binding the plurality of feed components 10 together in the process of moulding the feedstock 1. This can be advantageous as heat-activated binders can be used with conventional moulds (for e.g. polystyrene) without requiring any significant modifications.

In other examples, the binder 20 can have a different mechanism of activation. For example, the binder 20 can be chosen so that it is activated by pressure, chemical catalysis, radiation curing, and/or ultraviolet curing. In other examples, the binder 20 can be activated by one or more mechanisms that can be used as alternatives, or can be activated by a combination of mechanisms (e.g. both heat and pressure).

Furthermore, in some examples, the binder 20 can be activated before, during, or after the moulding process, as described in more detail herein.

In some examples, the binder 20 can be compostable and/or biodegradable. Because the feed components 10 can be compostable and/or biodegradable themselves, a compostable/biodegradable binder can be used to create a compostable/biodegradable feedstock 1 and/or formed products made from the feedstock 1. For example, the feedstock 1 and/or formed products made from the feedstock 1 can be home compostable and/or industrially compostable. For example, the feedstock 1 and/or products made therefrom may break down in 45 to 90 days in a typical home composting environment. In other examples, the feedstock 1 and/or products made therefrom may break down in up to 6 months in a typical home composting environment. In still further examples, the feedstock 1 and/or products made therefrom may break down in up to 12 months in a typical home composting environment.

The feedstock 1 and/or formed products made from the feedstock 1 can also be certifiably compostable and/or biodegradable. For example, the feedstock 1 and/or formed products made therefrom can comply with EN 13432, ASTM D6400, and/or AS4736 standards.

For example, the binder 20 can be compostable so that the binder 20, feedstock 1, and/or a formed product made from the feedstock can decompose rapidly. This can be particularly advantageous if feedstock 1 is being used, for example, to produce packaging materials as an alternative to polystyrene or other conventional plastic-based packaging materials. Conventional plastic-based packaging materials have a serious impact on the environment and present an ongoing waste management problem. The feedstock 1 can also be used to produce alternative materials for other applications where polystyrene is used.

The adhesion between feed components 10 due to the activation of binder 20 can create a much stronger bond between feed components 10 than would otherwise be achievable without a binder 20, meaning that feedstock 1 can be used to create formed products with higher strength and advantageous properties compared mycelium-based feedstocks which lack an activated binder. Furthermore, the material properties of the binder 20 can affect the material properties of formed products produced from feedstock 1 independently of the increased mechanical adhesion between feed components 10.

For example, the inclusion of binder 20 can increase the material strength of products formed at least partially from the feedstock 1. The formed product can have a higher impact resistance and/or higher shock absorption compared to formed products created using mycelium-based feedstocks which lack activated binders. Similarly, products formed from feedstock 1 can have higher acoustic and/or thermal insulation and can also or alternatively be hydrophobic and/or fire resistant.

The binder 20 can also affect the rate at which the feedstock 1 absorbs moisture from the atmosphere and/or the maximum moisture content of feedstock 1. In examples of feedstock 1 where the feed components 10 are first prepared before being coated with and/or impregnated by binder 20 (which are discussed herein), the feed components 10 can be dehydrated until they have substantially no residual moisture or a comparatively low level of moisture by weight. If the feed components 10 were exposed to ambient conditions for a sufficiently long time, they would gradually absorb moisture from the atmosphere. This would increase the density of the feed components 10 (thereby increasing expenses relating to shipping/handling of the feedstock) and would also reduce the shelf life of the feed components 10, as the relatively high moisture content could allow bacteria and other unintended fungi to contaminate the feed components 10.

To this end, the binder 20 can act to retard or prevent the uptake of atmospheric moisture by the feed components 10. For example, the binder 20 can act as an impermeable coating for the feed components 10, thereby preventing the feed components 10 from absorbing atmospheric moisture. The binder 20 can also prevent uptake of atmospheric moisture by plasticising and/or waterproofing the volume of the feed components 10 by impregnating the feed components 10.

As a result of the inclusion of binder 20, the feedstock 1 can have a moisture content that is substantially equal to or under 20% by weight; substantially equal to or under 10% by weight; and/or substantially about 0% by weight. Furthermore, the moisture content of the feedstock 1 may not increase with time. The shelf life of feedstock 1 can therefore be effectively indefinite due to the prevention of moisture uptake caused by binder 20.

Forming Products from Feedstocks

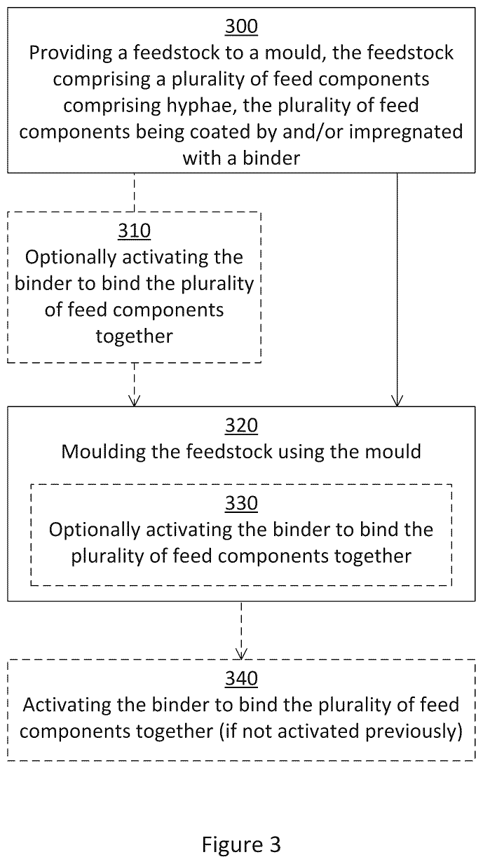

The feedstocks described herein can be suitable for moulding or forming into formed products. FIG. 3 depicts an example method of forming a product.

A feedstock is provided to a mould at 300. The feedstock can comprise a plurality of feed components comprising hyphae, the plurality of feed components being coated by and/or impregnated with a binder.

The feedstock is then moulded using the mould at 320. The mould can define a cavity or void which is occupied by the feedstock, thereby producing a formed product in the initial shape of the mould. For example, the mould can be a shape mould or a block mould.

The binder can be activated to bind the plurality of feed components together. The activation of the binder can take place before moulding of the feedstock (i.e. at 310), during moulding of the feedstock (i.e. at 330), or after the moulding of the feedstock (i.e. at 340).

The end result is a formed product at least partially moulded from the feedstock, the formed product comprising a plurality of feed components comprising hyphae and a binder, with the plurality of feed components being bound together by the binder. The shape and/or dimensions of the formed product can be determined by the mould according to the requirements of the formed product.

In some examples, the formed product can be post-processed after moulding. For example, if the feedstock is moulded using a block mould, then the formed product can be a large monolithic block of material. The moulded block of material can then be cut into sheets or other discrete components. A number of sheets or other components with bespoke shapes and dimensions can therefore be produced by post-processing the formed product without requiring a dedicated shape mould for each sheet/component.

The mechanism of activation of the binder (taking place at 310, 330, or 340) can depend at least partially on the characteristics of the binder. As has been described, the binder can be activated using, for example, heat, pressure, chemical catalysis, radiation curing, and/or ultraviolet curing.

For example, the binder can be activated by heat and/or pressure, and the mould to which the feedstock is provided at 300 can be a heated shape mould. The binder can then be activated at 330 during the moulding process 320 as the heated mould presses the feedstock. The formed product retrieved from the mould then comprises the binder that has been activated and a plurality of feed components that are bound together by the activated binder.

In other examples, the binder can be activated by heat but may be activated before or after the moulding of feedstock. For example, the feedstock 1 can first be moulded into a formed shape at 320 prior to the binder being activated at 340. The formed shape can then be retrieved from the mould and can be sintered after moulding by exposing the formed shape to sufficient heat for a sufficient duration to activate the binder within the feedstock at 340, thereby binding the feed components together. Alternatively, the binder can be activated at 310 prior to the feedstock being moulded, provided that the activated binder/feedstock is sufficiently malleable to take and hold the shape impressed by the mould at 320.

Binders which are not activated by heat can also be activated before, during, or after the moulding process at 310, 330, or 340, depending on the application of the feedstock. For example, if the binder is activated by ultraviolet curing, then the feedstock can be moulded into a formed shape at 320 prior to the binder being activated. The formed shape can then be retrieved from the mould and exposed to ultraviolet light at 340 to cure and activate the binder within the feedstock, thereby binding the plurality of feed components together. The binder can similarly be exposed to a chemical catalyst, radiation curing, and/or heat/pressure at 340 depending on the mechanism of activation of the binder.

### Preparation of Feed Components and Feedstocks

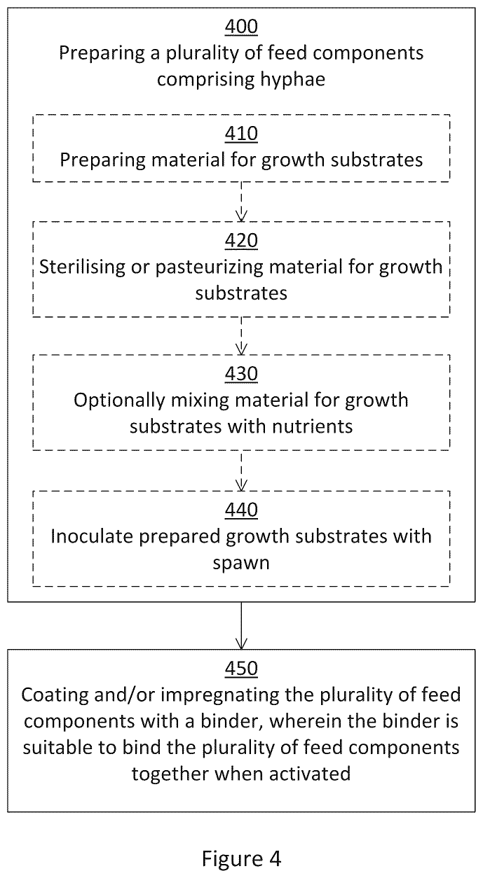

FIG. 4 depicts one example approach for producing a feedstock for moulding. A plurality of feed components comprising hyphae can be prepared at 400. The plurality of feed components can then be coated by and/or impregnated with a binder at 450. The binder can be suitable to bind the plurality of feed components together when activated.

In some examples, the feed components can be prepared at 400 by preparing growth substrates and allowing a fungus or fungi to colonise a growth substrate. In other examples, the feed components can be prepared at 400 using other techniques.

The growth substrate or substrates typically comprise organic matter and/or cellulose which can be prepared at 410. For example, the growth substrates can at least partially comprise shredded cardboard, straw, sawdust, hemp, hardwood or softwood chips/fibres or pulps thereof, organic materials, sugar cane/bagasse, textiles, and/or coffee grounds. Other organic waste products such as food waste, dried food/juice fibre, and/or husks or skins of foods (such as rice hulls, hemp hulls, coconut husks, etc) can also be at least partially incorporated into the material used to prepare the growth substrate.

The material(s) used to prepare the growth substrate can also optionally be mixed with one or more nutrients, as at 430. The nutrients can serve to accelerate mycelium growth of the colonising fungus. Mycelium can grow on a wide range of different nutrients including sugars, starches, lignin, fats, proteins, and nitrogen. The composition of the nutrients can depend, for example, on the materials used to form the growth substrate, the strain or species of fungus used to inoculate the growth substrates, and the end application of the growth substrate/feedstock.

The material used to prepare the growth substrate can be pasteurized or sterilized (using e.g. a combination of pressure and heat) at 420 and can be mixed together with the nutrients (if applicable) at 430 to produce a growth substrate. The growth substrate can then be inoculated with spawn comprising the fungus that will colonise the growth substrate at 440. Several different genus and species of fungus can be used to colonise the growth substrate, examples of which include turkey tail (Trametes versicolor), oyster mushrooms (Pleurotus sp.), and Ganoderma (Ganoderma sp.). The chosen fungus then colonises and grows throughout the growth substrate, creating a matrix of mycelium and hyphae. The spawn can typically grow for a period of 1 to 14 days, although this can depend on several factors including the dimensions/size of the growth substrate, the strain of the fungus, the material and nutrients comprising the growth substrate, etc.

Once the growth substrate has been adequately colonised by the chosen fungus, the growth substrate can be dehydrated to prevent further mycelial growth and to remove moisture from the growth substrate. For example, the growth substrate can be dehydrated in a dehydrator, an autoclave, a kiln, or a heated conveyor, although other dehydrating machines can also be used. The growth substrate may typically be exposed to temperatures of 60° C. to 100° C. for 4 to 10 hours, although the dehydration temperature and time period can depend at least on the size of the material to be cured, amongst other factors. The finished product after dehydration is a feed component comprising hyphae that is lightweight and has a low moisture content.

The feed components prepared at 400 can then be coated with and/or impregnated with a binder at 450. The binder can be suitable to bind the plurality of feed components together when activated, as discussed herein. Any of the binders discussed herein can be used to treat the feed components at 450. The binder can be applied to the feed components at 450 by (for example) spraying, rolling, submerging, or dousing the prepared feed components with the binder. This can take place with the binder at room temperature (if the binder is liquid at this temperature) or can take place at an elevated temperature if the binder is solid at room temperature.

In some examples, the prepared growth substrates can be formed into the final shape of the feed component before the growth substrate is inoculated at 440. For example, if sheetlike planar feed components are to be prepared at 400, then the growth substrate can be formed in the shape of a sheet before being inoculated with spawn at 440. The fungus can then colonise the growth substrate to produce a feed component having the shape of the growth substrate.

In other examples, the colonised growth substrate can be processed (e.g. mechanically processed) after inoculation or colonisation to produce the feed components. For example, if it is desired that the feed components will be in the shape of pellets, then a larger inoculated or colonised growth substrate (e.g. a sheet of growth material) can be processed (e.g. chopped or shredded) into multiple smaller pellet-sized substrates. These can then be dehydrated (once or if sufficiently colonised) to prepare the feed components at 400. Alternatively, a larger feed component can be processed into smaller feed components after dehydration in order to produce smaller feed components at 400.

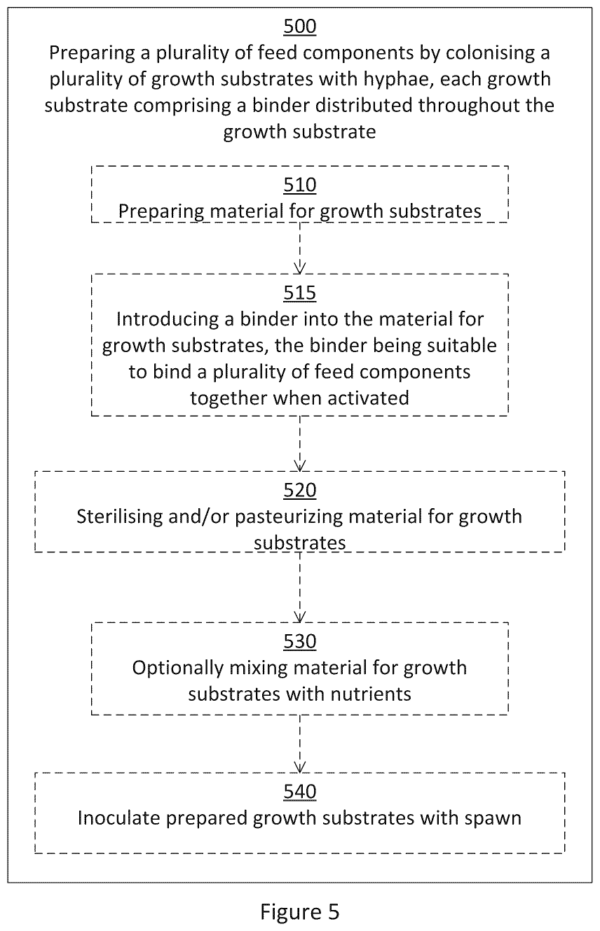

In other examples, feedstocks for moulding can be prepared by incorporating the binder into the growth substrate that is colonised by the fungus. FIG. 5 depicts an example of such a method.

A plurality of feed components can be prepared at 500 by colonising a plurality of growth substrates with hyphae. The growth substrates can be prepared using a growth substrate material (prepared at 510) optionally alongside a nutrient (at 530), in the same fashion as 410 and 430 with respect to FIG. 4. The growth substrates can also be formed into the shape of the final desired feed component if required. However, the preparation differs in that a binder is also introduced into the growth substrate material at 515, such that each prepared growth substrate of the plurality of growth substrates comprises a binder distributed throughout the growth substrate. The binder can be suitable to bind the plurality of feed components together when activated.

For example, the binder can be incorporated into the material for the growth substrate by mixing a solution of binder into the growth material and/or nutrients prior to inoculation at 540. For example, the binder can be a water soluble binder (e.g. PVA) which is dissolved and mixed throughout the growth material and/or nutrients. Alternatively, the binder can be distributed throughout the growth substrate in the form of particles of binder suspended in a matrix of growth substrate material and/or nutrient. For example, the binder can be granulated or pulverised before being mixed through the growth material and/or nutrients prior to inoculation by the fungus at 540.

After the growth substrates have been prepared such that the binder is distributed throughout each growth substrate, the growth substrates can be inoculated with spawn and colonised by fungus at 540 to prepare a plurality of feed components, as has been described with respect to 440 of FIG. 4. The colonised growth substrates can be dehydrated after sufficient fungal growth to reduce the moisture content of the feed components as has been described. Because the binder is already incorporated into the feed components at the time of dehydration, the dehydration process may be altered to prevent premature activation of the binder. For example, the dehydration process can take place at reduced temperatures and/or reduced pressures if the binder distributed throughout the growth substrate is activated by heat and/or pressure.

The final result is a feedstock comprising a plurality of feed components with the binder distributed throughout each feed component. The binder can be suitable to bind the plurality of feed components together when activated. The feed components do not need to be treated with additional binder due to the incorporation of binder throughout each feed component, although this can be done if desired (for example, to add a further coating of binder onto the feed components, or to include a different type of binder into the feedstock.)

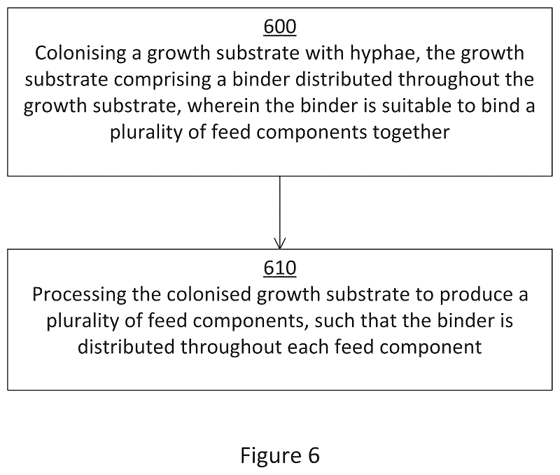

In other examples, a larger growth substrate comprising a binder can be colonised and processed to produce a plurality of feed components. An example of such a method is depicted in FIG. 6.

A growth substrate comprising a binder distributed therethroughout can be colonised with hyphae at 600. The growth substrate can be initially prepared as has been described with respect to 510, 515, 520, and 530 of FIG. 5 (namely, by mixing growth substrate materials (and optionally a nutrient) with a binder such that the binder is distributed throughout the growth substrate.) The growth substrate is then colonised with hyphae by, for example, inoculating the prepared growth substrate with fungal spawn (in a similar fashion to 540 with respect to FIG. 5).

After the growth substrate has been colonised by hyphae, the growth substrate can be processed at 610 to produce a plurality of feed components. For example, the growth substrate colonised at 600 can be a monolithic or comparatively large mass of colonised growth substrate, and the growth substrate can be mechanically chopped or processed into a plurality of smaller components (e.g. pellet-sized components.) These can then be dehydrated or treated as has been described to produce a plurality of feed components. Alternatively, the larger growth substrate can be dehydrated or treated prior to the processing of the growth substrate.

The end result is a feedstock that comprises a plurality of feed components, each of which includes a binder distributed therethroughout. The binder can be suitable to bind the plurality of feed components together when activated. The feedstock can then be used to mould a formed product as described herein.

### Continuous Production of Feed Components

In some examples, feed components for feedstocks can be created using a continuous process.

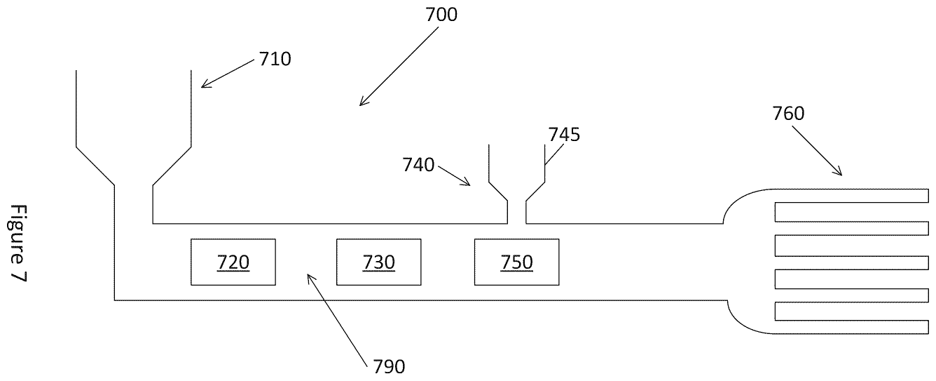

FIG. 7 depicts an example of an extrusion system 700 configured for the continuous production of feed components. The extrusion system 700 can include a hopper 710 which is used to feed material into extrusion line 790. The feed material can include material for growth substrates and nutrients for fungal growth. Binders can also be introduced into feed material/hopper 710 if desired. Although a single hopper 710 is depicted in FIG. 7, other extrusion systems can include multiple hoppers. Further examples of extrusion systems may use other means for introducing the feed materials (e.g. augers).

The extrusion system 700 can include one or more grinders or mixers 720 downstream of the hopper 710. The grinders or mixers 720 can be configured to grind or mix the feed materials that are introduced into the extrusion line 790 via hopper 710. Similarly, the extrusion system 700 can include an in-line sterilisation unit 730 which serves to sterilise the material provided to the extrusion line 790 by hopper 710. For example, the sterilisation unit 730 may comprise a heating unit, an autoclave, or other apparatus suitable to sterilise the material fed by the hopper 710. The grinders or mixers 720 and sterilisation unit 730 may be omitted in some examples of extrusion system 700 where pre-ground, pre-mixed, and/or pre-sterilised materials are exclusively provided to the extrusion line 790.

The extrusion system 700 can further comprise an inoculator 740 downstream of the sterilisation unit 730. The inoculator 740 can be configured to inoculate material provided to the extrusion line 790 with a fungal inoculum. For example, the inoculator 740 can comprise a feed unit 745 which stores a reservoir of spawn that can be progressively introduced into extrusion line 790. In some examples, the spawn can comprise grain spawn and/or liquid spawn. Spawn or other fungal inoculum introduced into the extrusion line 790 can be mixed with the feed material using a mixer 750 if desired. The inoculator 740 can also comprise a water source such as a spray jet (not depicted in FIG. 7) to increase the moisture of the inoculated feed material if required.

The extrusion system can further comprise a plurality of grow lines 760 downstream of the inoculator 740. The grow lines 760 can be configured to store colonised growth material which remains in-line as the fungal inoculum colonises the growth material. Hyphae can then grow throughout the colonised growth material as the colonised growth material progressively moves downstream through the grow lines 760.

The plurality of grow lines 760 can be configured so that once the colonised growth material residing within a given grow line 760 reaches the terminus of the grow line 760, hyphae have sufficiently grown so that the colonised growth material can be used as feed component material. The feed component material can then be extruded from the grow lines 760 and can be processed (e.g. chopped) into the desired dimensions of the feed component for the feedstock. The processed feed material can then be cured or dehydrated to produce a feed component. These may then be treated to coat and/or impregnated with a binder. Alternatively, the feed components may not require treatment with a binder if a binder has been introduced into the feed material through hopper 710.

The extrusion system 700 can therefore be used for the continuous production of a feed component. Growth material can be provided to the extrusion line through a hopper 710 or by other means. The growth material can then be colonised with a fungal inoculum provided by inoculator 740. With the colonised growth material remaining in-line, hyphae can be grown throughout the colonised growth material to produce a feed component material. The feed component material can then be extruded from the extrusion line 790.

In conclusion, the feedstocks for moulding that have been disclosed herein can be shipped to the point of production and processed within minutes at the mould, as they do not require any pre-growth period or growth within the mould. In comparison, other types of mycelium-based feedstocks must be injected or placed in the mould before the mycelium is grown, which takes time and resources.

The feedstocks can be lightweight and can have a very long (or even indefinite) shelf life due to the presence of binder within the feedstock. In comparison, other mycelium-based feedstocks that are processed directly at the mould can have high moisture content which can reduce shelf life and increase shipping and handling expenses. The binder can also increase the material strength of products formed from the feedstock and can alter or enhance other material properties of resulting products.

The feedstocks can be used to create alternative materials (such as packaging materials) in applications which traditionally use polystyrene or other plastics. However, the feedstocks and products produced therefrom can be biodegradable and/or compostable, which is a significant advantage over conventional polystyrene or plastic-based material. Furthermore, the feedstocks disclosed herein can be suited for use with existing moulds and/or tooling, including conventional moulds used to mould polystyrene. The feedstocks can also be used in other applications that do not traditionally use polystyrene.

While the present invention has been illustrated by the description of the examples thereof, and while the examples have been described in detail, it is not the intention of the Applicant to restrict or in any way limit the scope of the appended claims to such detail. Additional advantages and modifications will readily appear to those skilled in the art. Therefore, the invention in its broader aspects is not limited to the specific details, representative apparatus and method, and illustrative examples shown and described. Accordingly, departures may be made from such details without departure from the spirit or scope of the Applicant's general inventive concept.

1. A feedstock for moulding, comprising:
a plurality of feed components comprising hyphae; and
a binder,
wherein the plurality of feed components is coated by and/or impregnated with the binder, and the binder is suitable to bind the plurality of feed components together when activated.

2. The feedstock of claim 1, wherein the feed components at least partially comprise pellets, granules, a power, sheets, blocks, chips, balls, particles, fragments, shavings, and/or flakes.

3-13. (canceled)

14. The feedstock of claim 1, wherein the binder is a thermoplastic, polybutylene adipate terephthalate (PBAT), polyethylene terephthalate glycol-modified (PETG), polylactic acid (PLA), polybutylene succinate (PBS), polyphenylene sulfide (PPS), biopolyethylene, and/or a bioplastic.

15-21. (canceled)

22. The feedstock of claim 1, wherein the binder is activated by heat, pressure, chemical catalysis, radiation curing, and/or ultraviolet curing.

23-26. (canceled)

27. The feedstock of claim 1, wherein the binder is applied to the feed components via spraying, rolling, submerging or dousing.

28-30. (canceled)

31. The feedstock of claim 1, wherein the hyphae is grown by Trametes versicolor, Pleurotus sp., and/or Ganoderma sp..

32-33. (canceled)

34. The feedstock of claim 1, wherein the feedstock is compostable.

35-38. (canceled)

39. A method of forming a product, the method comprising:
providing a feedstock to a mould, the feedstock comprising a plurality of feed components comprising hyphae, the plurality of feed components being coated by and/or impregnated with a binder;
moulding the feedstock; and
before, after, or during the moulding of the feedstock, activating the binder to bind the plurality of feed components together.

40-41. (canceled)

42. The method of claim 39, wherein the binder is activated by heat, pressure, chemical catalysis, radiation curing, and/or ultraviolet curing.

43-45. (canceled)

46. A method of producing a feedstock for moulding, the method comprising:
preparing a plurality of feed components comprising hyphae; and
coating and/or impregnating the plurality of feed components with a binder,
wherein the binder is suitable to bind the plurality of feed components together when activated.

47. The method of claim 46, wherein the feed components at least partially comprise pellets, granules, a power, sheets, blocks, chips, balls, particles, fragments, shavings, and/or flakes.

48. The method of claim 46, wherein the binder is a thermoplastic, polybutylene adipate terephthalate (PBAT), polyethylene terephthalate glycol-modified (PETG), polylactic acid (PLA), polybutylene succinate (PBS), polyphenylene sulfide (PPS), biopolyethylene, and/or a bioplastic.

49. The method of claim 46, wherein the binder is activated by heat, pressure, chemical catalysis, radiation curing, and/or ultraviolet curing.

50. The method of claim 46, wherein the hyphae is grown by Trametes versicolor, Pleurotus sp., and/or Ganoderma sp..

51. The method of claim 46, wherein the binder is applied to the feed components via spraying, rolling, submerging, and/or dousing.

52-53. (canceled)

54. A method of producing a feedstock for moulding, the method comprising:
colonising a growth substrate with hyphae, the growth substrate comprising a binder distributed throughout the growth substrate; and
processing the colonised growth substrate to produce a plurality of feed components,
wherein the binder is distributed throughout each feed component, and
wherein the binder is suitable to bind the plurality of feed components together when activated.

55-61. (canceled)

62. A method of producing a feedstock for moulding, the method comprising:
preparing a plurality of feed components by colonising a plurality of growth substrates with hyphae, each growth substrate comprising a binder distributed throughout the growth substrate,
wherein the binder is distributed throughout each feed component, and
wherein the binder is suitable to bind the plurality of feed components together when activated.

63-68. (canceled)

69. A method for continuous production of a feed component, the method comprising:
providing growth material to an extrusion line;
colonising the growth material with a fungal inoculum;
with the colonised growth material remaining in-line, growing hyphae throughout the colonised growth material to produce a feed component material; and
extruding the feed component material.

70-71. (canceled)

72. A formed product at least partially moulded from a feedstock, the formed product comprising:
a plurality of feed components comprising hyphae; and
a binder,
wherein the plurality of feed components is bound together by the binder.

73-76. (canceled)
